(19) United States
(12) Patent Application Publication
(10) Pub. No.: US 2004/0049114 A1
(43) Pub. Date: Mar. 11, 2004

(54) Enclosure for a blood pressure cuff

(75) Inventor: Matthew L. Alesse, Hamlet of Ebenezer, NY (US)

Correspondence Address: Howard M. Elis, SIMPSON & SIMPSON, PLLC, 5555 Main Street, Williamsville, NY, 14221, US

(73) Assignee: Ethox Corporation, Buffalo, NY

(21) Appl. No.: 10/649,161

(22) Filed: Aug. 27, 2003

Related U.S. Application Data
(60) Provisional application No. 60/407,458, filed on Aug. 30, 2002.

(51) Int. Cl. A61B 5/02

(52) U.S. Cl. 600/490

(57) ABSTRACT

A reusable/disposable enclosure for an blood pressure cuff for a sphygmomanometer includes a pouch having an opening therein for holding a blood pressure cuff and further includes a flexible fastener operatively arranged to open the pouch and at least partially close the pouch when the blood pressure cuff is enclosed therein. The flexible fastener includes a first strip having at least one male rib portion extending therefrom and a second strip having at least two female rib portions extending therefrom, which form a channel for complementarily accepting the male rib portion. Thus, the first and second strips may be matingly engaged to one another for partially closing the enclosure. The enclosure is translucent and includes a fibrous outer surface and a fluid impermeable inner surface. The outer surface is comfortable to a user and the fluid impermeable surface prevents contamination and/or soiling of the blood pressure cuff contained therein.

### CROSS REFERENCE TO RELATED APPLICATIONS

The present application for patent claims the benefit under 35 USC §119(e) of U.S. Provisional Application No. 60/407,458, which provisional application is incorporated herein by reference.

### FIELD OF THE INVENTION

The present invention relates generally to an accessory for a device for measuring blood pressure, and more particularly, to a hygienic and sanitary enclosure for a blood pressure cuff of a sphygmomanometer that is reusable and/or disposable.

### BACKGROUND OF THE INVENTION

Most individuals at one time or another have had their blood pressure measured. Typically, measuring one's blood pressure occurs at a physician's office employing a sphygmomanometer. Generally, a sphygmomanometer comprises a ball pump, air delivery lines, a pressure gauge and an inflatable blood pressure cuff. The blood pressure cuff or BP cuff, is wrapped about one's bare arm and inflated and then deflated in the process of measuring one's blood pressure.

In settings such as a physician's office, it is usually standard practice for an unprotected, common blood pressure cuff to be reused to measure the blood pressure of a plurality of patients. While use of a blood pressure cuff by a plurality of patients may not be commonly associated with the transmission of body fluids or pathogens in a physician's office, in other settings, such as emergency rooms, trauma centers and the like, the common usage of a single blood pressure cuff by a plurality of patients can nevertheless present a health hazard. Indeed, in emergency rooms and trauma centers, for example, a blood pressure cuff may easily become soiled and/or contaminated with various types of body fluids, e.g., blood, microorganisms, bacteria, viruses, etc., which can ultimately lead to transmission of pathogens and disease between patients through use of a common blood pressure cuff.

To help prevent the transmission of pathogens between patients in emergency room type settings, disposable enclosures for blood pressure cuffs that can be discarded after each use have been developed. Such enclosures generally comprise a pouch or envelope with a large opening for introducing the BP cuff into the interior at one end, wherein the opening generally runs the length of the pouch, and often includes a closure mechanism for retaining the cuff in the interior, consisting of one or more adhesive strips.

Similarly, in hospital inpatient settings, it is also desirable to utilize a blood pressure cuff enclosure as a safety precaution to help prevent the transmission of disease and pathogens among the patient population. However, since inpatients often have their blood pressure checked several times a day during their stay, it is generally uneconomic to utilize a “fresh” previously unused cuff cover and dispose of the same after each patient's BP reading. Thus, reusable blood pressure cuff enclosures into which a blood pressure cuff may be inserted each time a patient's blood pressure is measured have been developed.

Closure mechanisms relying on adhesives, as a rule, are unsatisfactory in reusable BP cuff enclosures because of their inability to be resealed after the first opening of the seal for removal of the BP cuff. Consequently, reusable enclosures often comprise pouches with openings that are closeable via hook and loop type fasteners, such as VELCRO® brand fastener, or a zipper type closure mechanism (similar to the types of seals associated with plastic sandwich bags and/or plastic freezer bags). However, this inventor found that such closure means are not always reliable, and can often be cumbersome to use. For example, enclosures utilizing VELCRO® type hook and loop fasteners as closure means have a tendency to engage with and snag onto the corresponding hook and loop fastener tabs found on most blood pressure cuffs, when introducing the BP cuff inside the enclosure. Hence, it can become a tedious and difficult maneuver to insert a blood pressure cuff into these types of enclosures, often discouraging wide acceptance and usage by hospital personnel.

While zipper type seals seemingly offer the best solution for closing a blood pressure cuff cover, enclosures relying on such seals tend to undergo a separation or “curling” effect, i.e., the propensity of one strip of the zipper seal to separate from the other strip of the zipper seal as the enclosure is wrapped about a patient's arm or appendage. It is believed this curling effect may be attributed to a difference between the inner and outer circumferences of the zipper seal strips when the enclosure is rolled upon itself during the process of wrapping the enclosed BP cuff around a patient's arm.

Accordingly, there is a need for an improved, more reliable and convenient to use blood pressure cuff enclosure which is reusable so it can be assigned to a single patient at the time of admission and reused for the period of the patient's stay, and also sufficiently economic so that it is also disposable after a single use or after multiple uses.

### SUMMARY OF THE INVENTION

The present invention broadly comprises a reusable and/or disposable enclosure for a blood pressure cuff for a sphygmomanometer. In a preferred embodiment, the enclosure comprises a pouch having an opening therein for holding a blood pressure cuff. The enclosure includes a flexible fastener, e.g., a zipper type seal of the type typically associated with a resealable sandwich bag and/or a resealable freezer bag, that is operatively arranged to open the pouch and at least partially close the pouch when the blood pressure cuff is enclosed therein. In a particularly preferred embodiment, the enclosure includes a flexible fastener, e.g. a zipper seal, having a first strip and a second strip. The first strip includes at least one male rib portion extending therefrom and along its length. The second strip including at least two female rib portions extending therefrom and along its length. The female rib portions of the second strip form a channel for complementarily accepting the male rib portion of the first strip such that the first strip and the second strip may be matingly engaged with one another to partially close the enclosure. The enclosure may be constructed from at least one ply of a translucent polyolefin comprising a spun, non-woven, fibrous outer surface and a nonporous, liquid impermeable inner surface. The spun, non-woven, fibrous outer surface is operatively arranged for direct contact with a user's skin such that the blood pressure cuff enclosure is comfortable to the user and effectively facilitates evaporative cooling at the surface of the skin. The inner surface is, preferably, impermeable to fluids for purposes of preventing contamination and/or soiling of the blood pressure cuff contained therein and/or to prevent a contaminated blood pressure cuff from contacting a patient. The enclosure also prevents patients from viewing a potentially unsightly and/or soiled blood pressure cuff.

Thus, a primary object of the present invention is to provide a highly economic “reposable” (i.e., reusable and disposable) blood pressure cuff enclosure with an improved flexible fastener for securing a blood pressure cuff therein while also allowing the unimpeded insertion and removal of a blood pressure cuff to and from the enclosure.

These and other objects, features and advantages of the invention will become readily apparent to one having ordinary skill in the art upon study of the following detailed description in view of the drawings and appended claims.

### BRIEF DESCRIPTION OF THE DRAWINGS

FIG. 1 shows a perspective view of a blood pressure cuff enclosure of the present invention wrapped about a patient's arm;

FIG. 2 is a view of the bottom panel (panel in contact with a user's appendage) of a blood pressure cuff enclosure according to the present invention, the enclosure including a blood pressure cuff therein;

FIG. 3 is a view of a top panel of a blood pressure cuff enclosure according to the present invention;

FIG. 4 is a sectional view of the present invention taken generally along line 4-4 of FIG. 3, illustrating the thickness of the flange of the flexible fastener means; the enclosure including a blood pressure cuff therein;

FIG. 5 is a sectional view of the present invention taken generally along line 5-5 of FIG. 3;

FIG. 6 is a perspective view of a blood pressure cuff enclosure including enlarged surface areas to illustrate the non-woven, fibrous outer surface of an embodiment of the present invention;

FIG. 7 is a view of the bottom panel (panel in contact with a user's appendage) of a blood pressure cuff enclosure according to the present invention comprising a fibrous outer surface, the enclosure including a blood pressure cuff therein;

FIG. 8 is a view of the top panel of a blood pressure cuff enclosure according to the present invention comprising a fibrous outer surface, the enclosure including a blood pressure cuff therein;

FIG. 9 is a sectional view of the present invention taken generally along line 9-9 of FIG. 8;

FIG. 10 is a sectional view of the present invention taken generally along line 10-10 of FIG. 8.

### DETAILED DESCRIPTION OF THE PREFERRED EMBODIMENT

At the outset, it should be appreciated that like reference numbers on different drawing figures represent identical structural elements. It should also be appreciated that, while a number of different embodiments and variations of the present invention are shown in the various drawings, the invention as claimed is not intended to be limited to these specific embodiments, as the claims define a broader invention that can take many different shapes and structures. In the description that follows, the term “reposable” is intended to refer to reusable and, optionally, disposable qualities of the enclosure of the present invention. In the description and claims that follow, the term “curling” is intended to mean the propensity of the male and female strips of a flexible fastener, e.g., a zipper seal of a type typically associated with a resealable plastic sandwich bag or freezer bag, to separate from one another when the flexible fastener is rolled or wrapped upon itself, e.g., when the enclosure and the flexible fastener are wrapped about an individual's arm for purposes of measuring blood pressure.

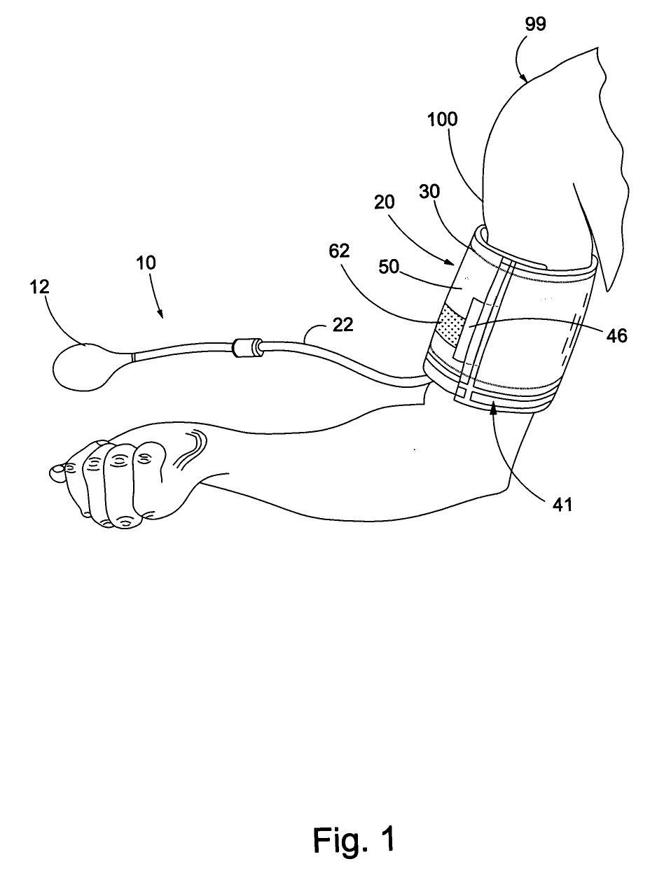

Adverting now to the figures, in FIG. 1 enclosure 30 of the present invention is shown in association with sphygmomanometer 10, of which blood pressure cuff 20 is a component thereof. The blood pressure cuff enclosure and the blood pressure cuff are shown wrapped about a patient's arm for purposes of measuring the patient's blood pressure. A typically sphygmomanometer 10 generally includes pump 12, pressure gauge (not shown), air delivery tube 22 and blood pressure cuff 20. As is generally the case, the blood pressure cuff of a sphygmomanometer is usually rectangular in shape and comprises hook and loop tab fasteners proximate the terminal ends of the blood pressure cuff. The blood pressure cuff, thus, may be wrapped about a patient's arm and its terminal ends secured to one another by means of the hook and loop fasteners. Once secured about arm 100 of patient 99, the blood pressure cuff is then inflated by means of the pump, which passes air to the blood pressure cuff via air delivery tube 22 until blood flowing through a patient's artery has ceased. Once a patient's arterial blood flow has ceased, further pressurization of the blood pressure cuff is also ceased. Thereafter, the blood pressure cuff is allowed to slowly deflate so that the systolic and diastolic blood pressures of the patient may be measured. It should be appreciated by those having ordinary skill in the art that, while an enclosure for a manually operated sphygmomanometer is disclosed herein, the enclosure of the present invention may be used with sphygmomanometers that are automatically inflated and/or deflated.

### Structure of a Preferred Embodiment

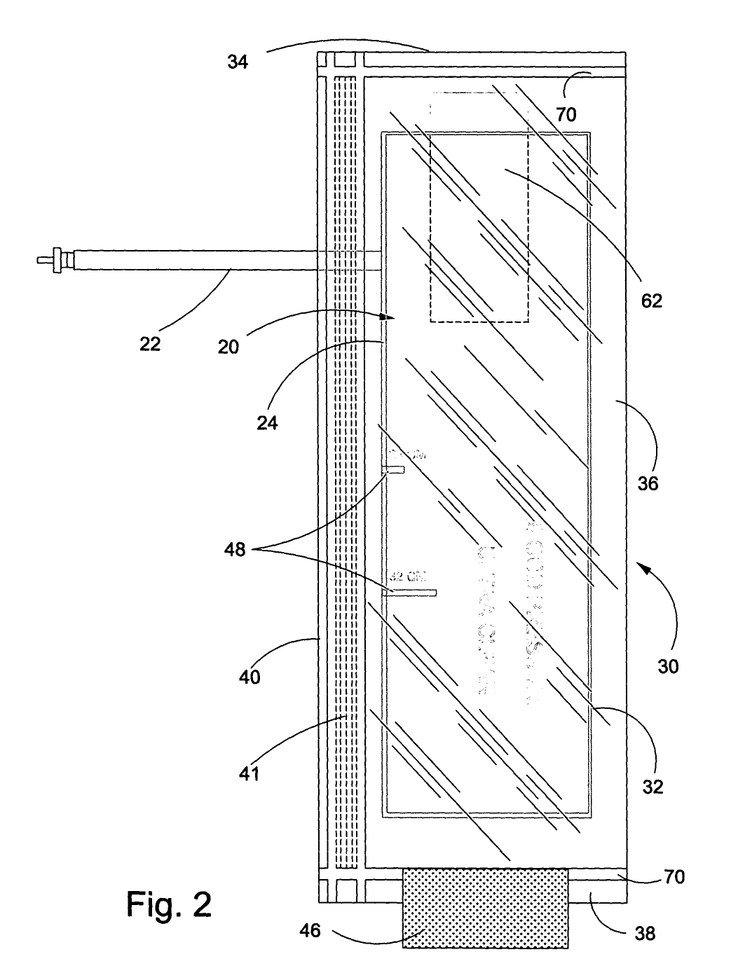

Referring more specifically now to FIGS. 2-5; enclosure 30 of the present invention generally forms a pouch. The pouch is generally in the manner of an envelope and is operatively arranged to receive an blood pressure cuff of a sphygmomanometer therein such that the blood pressure cuff does not become contaminated by bodily fluids or become soiled. In a preferred embodiment, the enclosure is made from a single sheet of enclosure material that has been folded in half and joined along its edges to form top panel 50 and bottom panel 32. When folded in such manner enclosure 30 comprises first edge 34, second edge 36 (fold edge), third edge 38 and fourth edge 40.

In the embodiment illustrated in the drawings, the top and bottom panels are secured to one another along first edge 34 and third edge 38 to form seals 70. Seals 70 extend substantially along the first and the third edges between the second and fourth edges. In a preferred embodiment, seals 70 are preferably formed by ultrasonically welding the first and third edges of the top and bottom panels to one another. In this manner an enclosure having interior 74 is provided in which blood pressure cuff 20 may be secured. It should be appreciated that other means for securing the first and third edges to one another are contemplated and may include but are not limited to: heat welding, adhesives, stitching, etc. It should also be appreciated by those having ordinary skill in the art, that while a preferred embodiment comprises a single piece of sheet material that has been folded upon itself to form a pouch or envelope, substantially similar results may be achieved by securing two or more independent sheets together to form a pouch or envelope. Obviously, other methods of folding the enclosure and/or forming a pouch are contemplated and intended to be encompassed by the present claims and disclosure, as are other shapes of pouch.

Disposed proximate first edge 34 and fixedly secured to top panel 50 is a tab section of loop 62. Disposed proximate third edge 38 of bottom panel 32 is tab section of hook 46. Loop material 62 is provided for binding with hook material 46 when the enclosure and blood pressure cuff are wrapped about a patient's arm or appendage. It should be appreciated that loop material 62 comprises a length such that the enclosure may be adjusted about the arm or appendage for a substantially snug fit. It should also be appreciated by those having ordinary skill in the art that while we disclose a enclosure comprising hook material 46 and loop material 62 disposed on bottom and top panels, 32 and 50, respectively, the locations of the hook and loop material could be reversed to achieve like results.

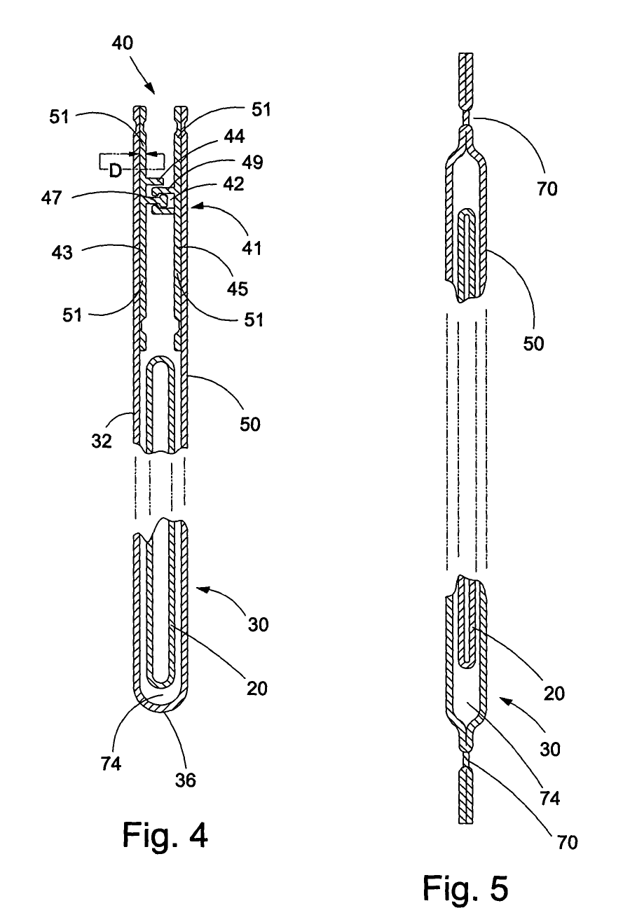

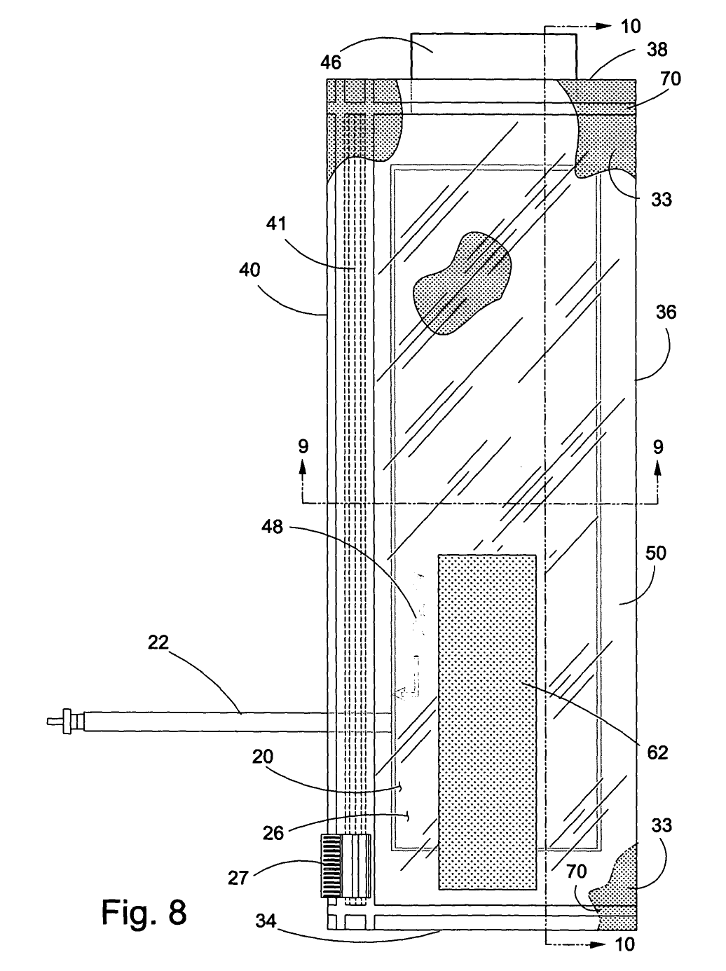

As shown in FIGS. 2-4, fourth edge 40 of the enclosure of the present invention includes flexible fastener 41. Flexible fastener 41 comprises means for resealably opening and closing the enclosure such that a blood pressure cuff may be readily inserted or removed therefrom for the term of a patient's stay. In a preferred embodiment, flexible fastener 41 comprises first strip 43 and second strip 45. First strip 43 is secured to an inner side of bottom panel 32 proximate fourth edge 40 and second strip 45 is secured to an inner surface of top panel 50 proximate fourth edge 40. Each of first strip 43 and second strip 45 extend substantially along the entire length of fourth edge 40 and are disposed opposite one another. Extending from first strip 43 and substantially along the length of fourth edge 40 is at least one male rib 47 and guide means 44. Similarly, extending from second strip 45 and substantially along the length of fourth 40 edge are at least two parallel and spaced female ribs 49. At least two female ribs 49 form channel 42 therebetween, which channel is configured for complementarily accepting male rib 47 therein. Hence, the flexible fastener, and the enclosure, may be closed by means of mating the male rib with the female channel with or without the aid of a zipper clasp means 27 (See FIG. 8) for opening and closing the enclosure. It should be appreciated that first strip 43 and second strip 45 of flexible fastener 41 are secured to the enclosure by flanges 51 which may be secured to the bottom and top panels by any appropriate means including but not limited to: heat welding, ultrasonic welding, adhesives, stitching, etc. The inventor discovered that flexible fasteners comprising thick flanges have a tendency to “curl” and open when the enclosure is wrapped about a patient's arm and further found that the problem of curling can be rectified by employing flanges that are sufficiently thin such that the flexible fastener does not have a propensity to curl when the enclosure is closed. More specifically, FIG. 4 illustrates that a preferred thickness, D, of flange 51 of a flexible fastener between 2 and 12 mils, and more preferably, between 4 and 8 mils will eliminate curling and separation of the flexible fastener during the closing and wrapping process. Additionally, other types of flexible fastener, such as hook and loop fasteners, adhesives, etc., are contemplated herein but have been found to be generally ineffective as discussed supra.

Hook and loop type flexible fasteners have a tendency to bind with the hook and loop fasteners of the sphygmomanometer blood pressure cuff such that it can be difficult to insert and remove the blood pressure cuff from the enclosure. Similarly, because the enclosure of the present invention is configured for being reposable, that is, both reusable and disposable, resealable adhesives for opening and closing the enclosure have been found to be largely ineffective as well. Adhesives may become spent or soiled such that the enclosure can no longer be closed or the adhesives may be too strong such that damage to the enclosure may occur when the enclosure is opened for removal of the blood pressure cuff. The improved flexible fastener of the present invention will obviate this problem.

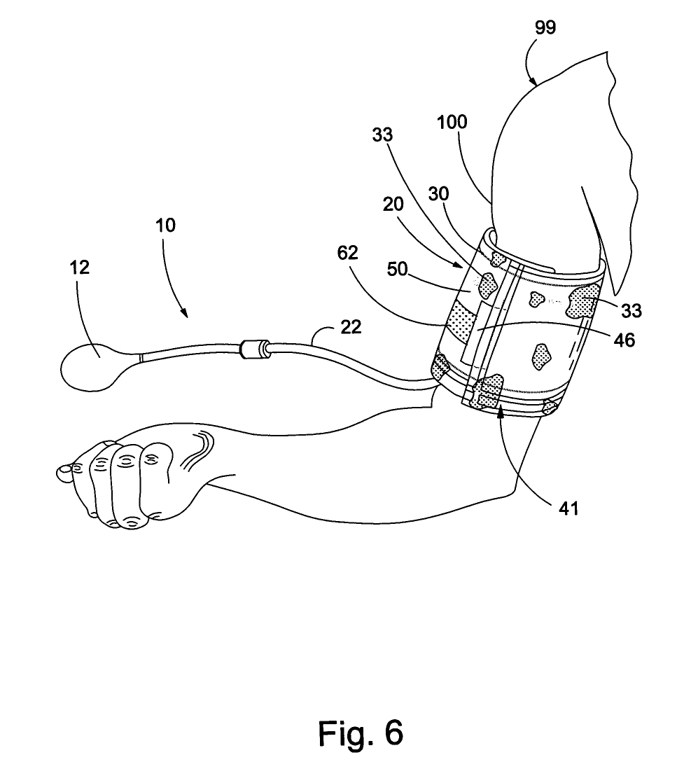

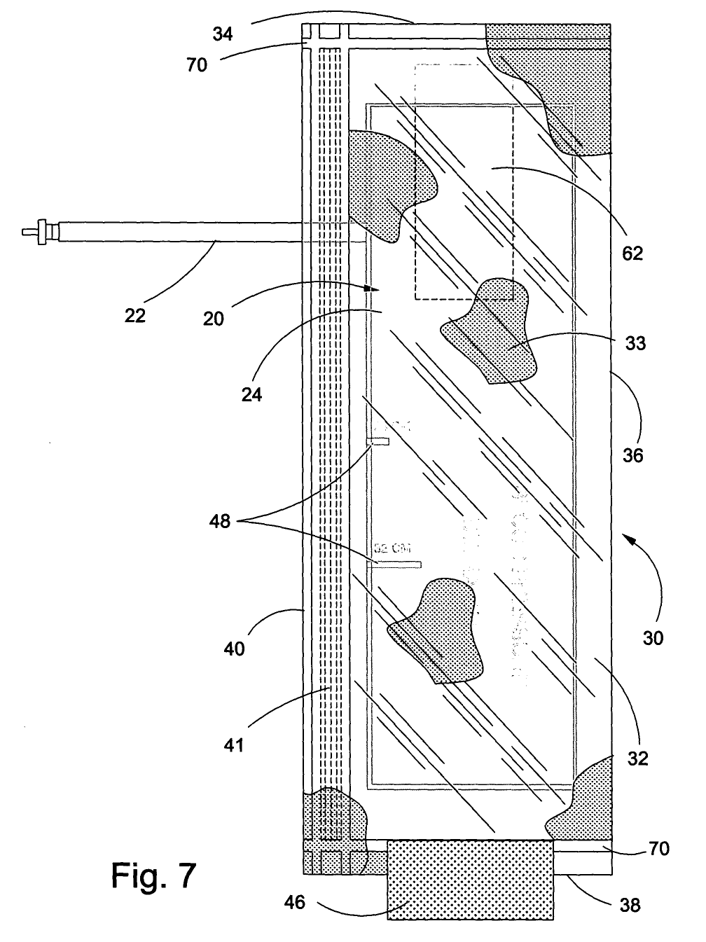

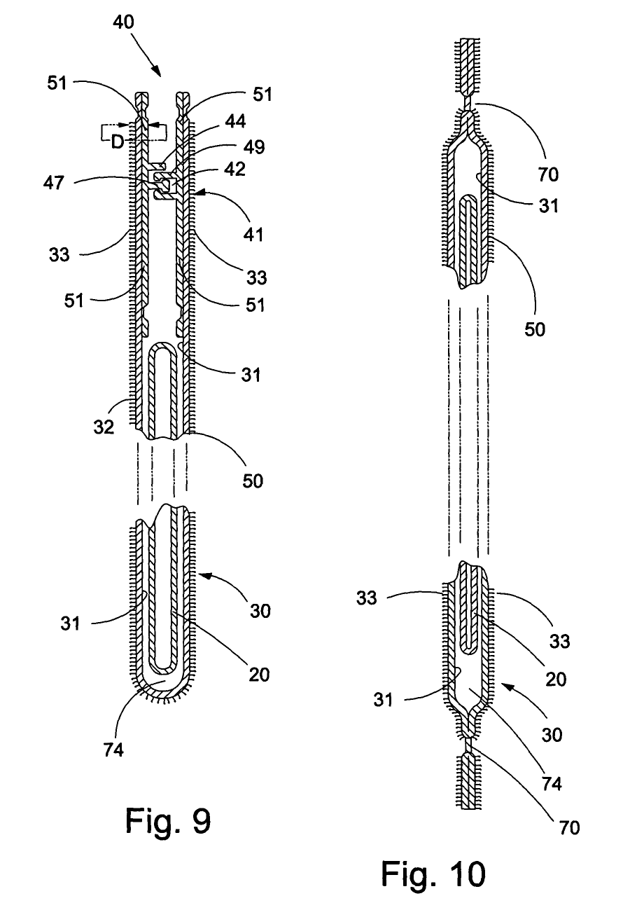

Referring now to FIGS. 6-10, while the sheet material comprising the top and bottom panels of the enclosure may be constructed from a wide range of materials, a preferred embodiment of the enclosure is constructed from at least a single ply of sheet material that is impermeable to fluids while simultaneously being comfortable to the user. Inner surface 31 of the enclosure comprises at least a single ply of polyolefin film, e.g. polyethylene, which is nonporous to fluids, whereas outer surface 33 has a fibrous texture. In a preferred embodiment, the fibrous texture of the outer surface is provided by a plurality of spun bonded, non-woven fibers, which create a flocked material surface that is soft to the touch. It should be appreciated that while FIGS. 6-10 illustrate outer surface 33 as comprising enlarged “patches” of the fibrous material, the entire outer surface of the enclosure comprises a fibrous texture and the “patches” shown in the drawings are provided solely for illustration. The inner continuous film surface of the enclosure primarily serves as a fluid and pathogen barrier and is impermeable to blood, bacteria, viruses and the like. Thus, the inner surface of the enclosure prevents the blood pressure cuff from becoming contaminated and/or soiled. On the opposite side of the film ply, the fibrous outer surface of the enclosure is soft and allows ambient air to contact the patient's skin, which facilitates evaporation at the skin surface making the enclosure more comfortable to the patient. The fibrous outer surface also prevents any chafing of the skin that may typically occur. Additionally, while a preferred embodiment of the present invention comprises section of loop material 62 for binding hook material 46, it should be appreciated that where the outer surface of the enclosure is sufficiently fibrous, hook material 46 may be directly bound to the outer surface of the enclosure such that the tab section of loop may be dispensed with.

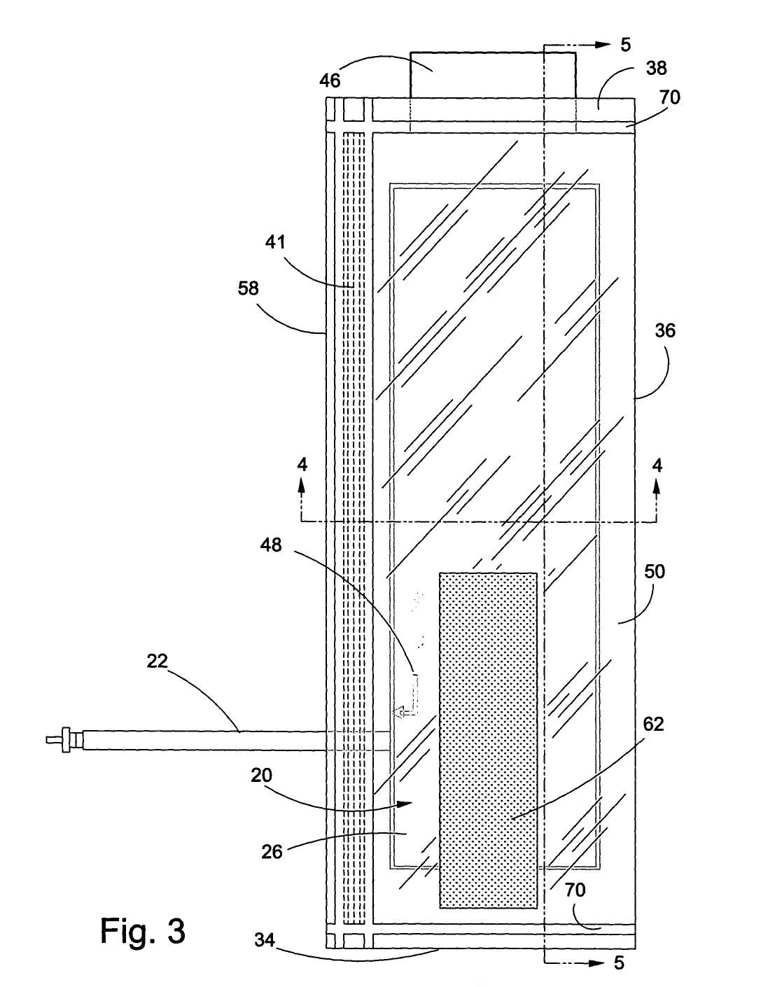

Referring now to FIGS. 1-10, in a preferred embodiment, the sheet material of the enclosure is translucent such that written indicia appearing on the blood pressure cuff contained therein may be read when the enclosure is engaged with the surface of the blood pressure cuff. For example, written indicia 48 on first edge 24 of the blood pressure cuff 20 may be read through translucent bottom surface 32 and may comprise terms such as “24 cm” and “32 cm,” as shown in FIG. 2. Second edge 26 of the blood pressure cuff 20 (FIG. 3) may also comprise written indicia 48, for example, the term ARTERY. The written indicia are provided for assisting the healthcare provider to align the blood pressure cuff in accordance with the location of a patient's arteries. It should be appreciated that while the enclosure of the present invention is translucent such that written indicia on the surface of the blood pressure cuff may be viewed, the translucent material is simultaneously sufficiently opaque such that it is difficult for a patient to see contaminants such as dirt, stains, blood, etc. on the blood pressure cuff. Hence, the enclosure of the present invention provides additional comfort to the patient in that the patient is not likely to view dirt, stains, blood and the like on the blood pressure cuff. It should be readily apparent to those having ordinary skill in the art that the written indicia on the blood pressure cuff 20 may comprise virtually any information that is helpful to the healthcare practitioner and is not limited to the written indicia shown in the figures. In a particularly preferred embodiment, the enclosure of the present invention further comprises means for distinguishing one enclosure from another enclosure such that the same enclosure is not used for two different patients. Appropriate means for distinguishing one enclosure from another include labels, color-coding, etc.,

### Use of a Preferred Embodiment

To use the enclosure of the present invention, the healthcare practitioner need merely open the flexible fastener and insert the blood pressure cuff therein. Thereafter, the flexible fastener may be partially closed by mateably securing the male rib with the channel formed by the female ribs; it should be appreciated that the flexible fastener remains partially closed when the blood pressure cuff is secured within the enclosure for purposes of accommodating the passage of air delivery tube 22 to the sphygmomanometer. Once secured within the enclosure, the written indicia on the blood pressure cuff may be viewed through the translucent enclosure material such that the blood pressure cuff may be aligned with a patient's artery. The enclosure and blood pressure cuff may then be wrapped about and secured to the arm or appendage of a patient by means of hook material 46 and loop material 62 fasteners. The blood pressure cuff is then inflated until blood within the artery has ceased flowing. The blood pressure cuff is then deflated to measure the patient's systolic and diastolic blood pressures. Once completed, the enclosure and blood pressure cuff may be removed from the patient's arm or appendage. Thereafter, the blood pressure cuff may be removed from within the enclosure and inserted in a new enclosure for purposes of measuring another individual's blood pressure. The enclosure used to measure a first patient's blood pressure may be disposed of, or it may be saved for purposes of taking another blood pressure reading from that same patient at a later time. Hence, the enclosure of the present invention is configured to be both reusable and disposable.

Thus, it is seen that the objects of the present invention are efficiently obtained, although modifications and changes to the invention should be readily apparent to those having ordinary skill in the art, and these modifications are intended to be within the spirit and scope of the invention as claimed.

What is claimed is:

1. An enclosure for a blood pressure cuff comprising:
a pouch having an opening therein to hold said blood pressure cuff; and,
a flexible fastener,
said flexible fastener having a thickness sufficiently thin so as to prevent curling when said enclosure is wrapped upon itself; said flexible fastener adapted to open said pouch and at least partially close said pouch when said blood pressure cuff is enclosed therein.

2. The enclosure of claim 1 wherein said flexible fastener comprises a first strip having at least one male rib portion extending therefrom and a second strip including at least two female rib portions extending therefrom, said female ribs forming a channel for complementarily accepting said male rib portion therein such that said first strip and second strip may be matingly engaged

3. The enclosure of claim 1 wherein said flexible fastener comprises a flange having a thickness between 2 and 12 mils.

4. The enclosure of claim 3 wherein said flexible fastener comprises a flange having a thickness between 4-8 mils.

5. The enclosure of claim 2 wherein said flexible fastener further comprises a zipper clasp.

6. The enclosure of claim 1 wherein said enclosure comprises at least a single ply of polyolefin film having an inner surface that is impermeable to liquids.

7. The enclosure of claim 6 wherein said enclosure comprises at least a single ply of polyolefin film having an outer surface with a fibrous texture.

8. The enclosure of claim 7 wherein said flexible fastener is disposed proximate said opening.

9. The enclosure of claim 8 wherein said flexible fastener further comprises:
a first strip including at least one male rib portion extending therefrom; and,
a second strip including at least two female rib portions extending therefrom, said female ribs forming a channel for complementarily accepting said male rib portion therein such that said first strip and second strip may be matingly engaged.

10. The enclosure of claim 1 wherein said enclosure is translucent.

11. The enclosure of claim 8 wherein said enclosure is translucent.

12. The enclosure of claim 8 wherein said pouch is formed by folding said at least a single ply of polyolefin film upon itself.

13. The enclosure of claim 12 wherein the at least one edge of said pouch comprises an ultrasonic weld.

14. The enclosure of claim 1 further comprising securing means for fastening said enclosure to itself when rolled upon itself.

15. The enclosure of claim 14 wherein said securing means comprises hook and loop.

16. The enclosure of claim 1 comprising a length sufficient such that said enclosure may be wrapped about an appendage.

17. The enclosure of claim 1 further comprising means for distinguishing said enclosure from another enclosure.

18. The enclosure of claim 17 wherein said means for distinguishing comprises a label secured to a top panel of at least one of said enclosures.

19. A method of using a blood pressure cuff for purposes of minimizing contamination between at least two patients using a single blood pressure cuff, said method comprising:
providing at least two enclosures having openings therein to hold said blood pressure cuff for said at least two patients; said at least two enclosures having flexible fasteners having a thickness sufficiently thin so as to prevent curling when said enclosures are wrapped upon themselves; said flexible fastener adapted to open said enclosures and at least partially close said enclosures when said blood pressure cuff is enclosed therein;
securing said blood pressure cuff within a first enclosure and measuring the blood pressure of a first patient;
removing said blood pressure cuff from said first enclosure; and,
securing said blood pressure cuff within a second enclosure and measuring the blood pressure of a second patient.
removing said blood pressure cuff from said second enclosure; and,

20. The method of claim 19 further comprising securing said blood pressure cuff within said first enclosure and re-measuring the blood pressure of said first patient.

21. The method of claim 20 wherein said flexible fasteners comprise:
a first strip including at least one male rib portion extending therefrom; and,
a second strip including at least two female rib portions extending therefrom, said female ribs forming a channel for complementarily accepting said male rib portion therein such that said first strip and second strip may be matingly engaged.

22. The method of claim 21 wherein said flexible fasteners each comprise a flange having a thickness between 2 and 12 mils.

23. The method of claim 22 wherein said flexible fastener has a flange having a thickness between 4-8 mils.

24. The method of claim 20 further comprising providing means for distinguishing said at least two enclosures from one another.

25. The method of claim 24 wherein said means for distinguishing comprises a label secured to a top panel of at least one of said enclosures.
